(19) United States
(12) Patent Application Publication
(10) Pub. No.: US 2005/0277805 A1
(43) Pub. Date: Dec. 15, 2005

(54) Fecal incontinence assistance device

(76) Inventor: Daniel M. Hatton, Follett, TX (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/382,561

(22) Filed: Mar. 6, 2003

(51) Int. Cl. A61F 2/00

(52) U.S. Cl. 600/29

(57) ABSTRACT

The present disclosure provides a reusable medical device including a disposable portion and a processor. The disposable portion includes a probe capable of insertion into an opening of the human body for detecting a condition within the body and a memory associated with the probe for storing a value representing an amount of time that the probe has been in use. The processor is electrically connected to the disposable portion and reads and updates the value stored within the memory. The processor further alerts the human or a caretaker of the presence of the condition and/or the expiration of the disposable portion.

### FIELD OF THE INVENTION

The present invention relates to fecal incontinence assistance devices. More specifically, it relates to a fecal incontinence assistance device with a time of use limitation function.

### BACKGROUND OF THE INVENTION

Many personal medical devices are designed to be used for a limited amount of time or a limited number of occurrences of a particular event. In a supervised setting such as a hospital or nursing home, requiring caregivers to follow an established procedure is the normal method to guaranty that those devices are not used beyond their expiration. In that case, the hospital or nursing home must rely on written or computerized care logs to verify that such devices are changed according to their required schedules. In the case of unsupervised care, where patients use the devices themselves, there is no way to ensure that the devices are changed according to their required schedules.

### SUMMARY OF THE INVENTION

The present invention provides a reusable medical device comprising a disposable portion and a processor. The disposable portion comprises a probe capable of insertion into an opening of the human body for detecting a condition within the body and a memory associated with the probe for storing a value representing an amount of time that the probe has been in use. The processor is electrically connected to the disposable portion and reads and updates the value stored within the memory. The processor further alerts the human or a caretaker of the presence of the condition.

### BRIEF DESCRIPTION OF THE DRAWINGS

FIG. 1 is a perspective view of a disposable catheter according to an embodiment of the of the present invention;

FIG. 2 is a perspective view of FIG. 1 having a fecal guard and a filter tip installed upon the probe according to an embodiment of the of the present invention;

FIG. 3 is a section view of a catheter probe according to an embodiment of the of the present invention;

FIG. 4 is a perspective view of a memory associated with a disposable catheter according to an embodiment of the of the present invention;

FIG. 5 is a perspective view of a memory encapsulated in epoxy associated with a disposable catheter according to an embodiment of the of the present invention;

FIG. 6 is a front perspective view of a processor according to an embodiment of the of the present invention;

FIG. 7A is a rear perspective view of a processor without a battery door attached according to an embodiment of the of the present invention;

FIG. 7B is a rear perspective view of a processor with a battery door attached according to an embodiment of the of the present invention;

FIG. 8 is a schematic view of a disposable catheter and processor according to an embodiment of the of the present invention;

FIGS. 9A, 9B, and 9C are perspective views of fecal guards according to an embodiment of the of the present invention; and

FIG. 10 is a perspective view of a filter tip according to an embodiment of the present invention.

### DESCRIPTION OF THE PREFERRED EMBODIMENT

While the invention is susceptible of embodiment in many different forms, there is described in detail preferred embodiments of the invention. It is to be understood that the present disclosure is to be considered only as an example of the principles of the invention. This disclosure is not intended to limit the broad aspect of the invention to the illustrated embodiments. The scope of protection should only be limited by the claims.

Disclosed is a device for insertion into a human body for blocking an opening of the human body and alerting the person, or an individual caring for the person, when a quantity of effluent has accumulated near the device within the opening. The device is further capable of alerting the person or a caretaker to remove the device to allow the effluent to exit the body. Furthermore, the device has the capability to alert the person when the device should be replaced by the user or caretaker because it has reached a limit beyond which it may no longer be used safely, such that risk of infection or malfunction is minimized.

In one particular application the device may be used as a fecal incontinence monitor. The fecal incontinence monitor may be inserted in the rectum to block the accidental passage of feces. The device further detects when a quantity of feces has accumulated in the rectum near the monitor to notify the patient or caregiver when fecal matter is present so that it may be expelled at the next opportunity.

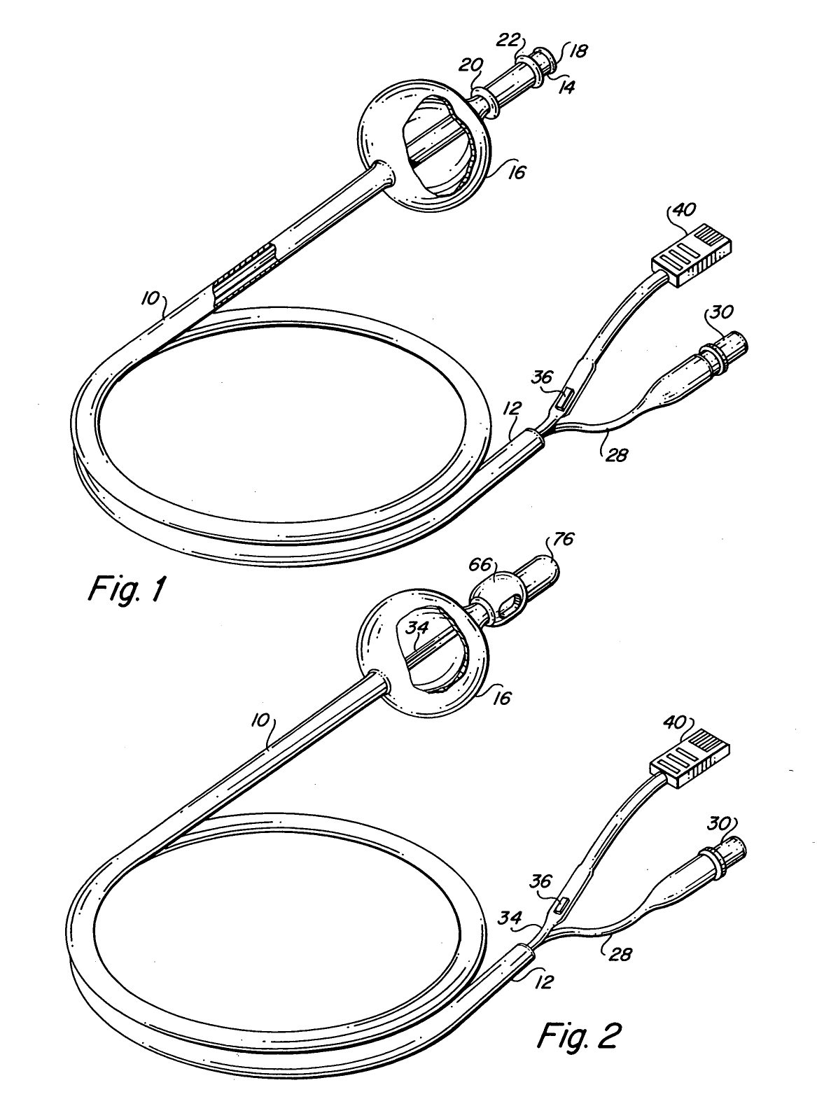

In this regard and referring to FIGS. 1, 2, & 3, the device comprises a silicone catheter tube 10 having a proximal end 12 and a distal end 14. Near the distal end 14 of the catheter tube 10 is an inflatable balloon 16. Beyond the inflatable balloon 16 is a probe 18. The probe 18 is made from a rigid material and comprises a hollow cylinder having two annular exterior barbed ribs 24, 26 and two journals 20,22. The silicone tube 10 is placed onto the probe 18 by sliding the tube 10 over barbed rib 26. The probe 18 further defines two metal electrical contacts 25 molded into the probe 18 such that the contacts 25 protrude through the probe 18 on opposite sides, extend into the interior of the probe 18 and through an opening at an end of the probe 18. The contacts 25 are attached to electrically conductive wire 34 located within the catheter tube 10. As a result, if a potential difference is applied to the contacts 25, when an electrically conductive fluid comes in contact the electrical contacts 25 a complete circuit is accomplished through the fluid, and in this manner comprise a moisture detector.

The balloon 16 is attached to and is in fluid communication with a hollow air supply tube 28 within the catheter tube 10. In FIG. 1, the balloon 16 is shown in its inflated state. When in its uninflated state, the balloon 16 has an outer diameter generally no greater than that of the catheter tube 10.

The hollow air supply tube 28 is further in fluid communication with a check valve 30. The check valve 30 is biased to a normally closed state wherein air within the air supply tube 28 is trapped within the tube 28. Furthermore, the balloon 16 is designed to allow air to permeate through the balloon 16 such that an inflated balloon will slowly deflate. In this manner, decubitus ulcers caused by the pressure of the balloon 16 cutting off blood supply to a portion of the rectum are avoided. However, this does require the user to occasionally reinflate the balloon 16, as described below.

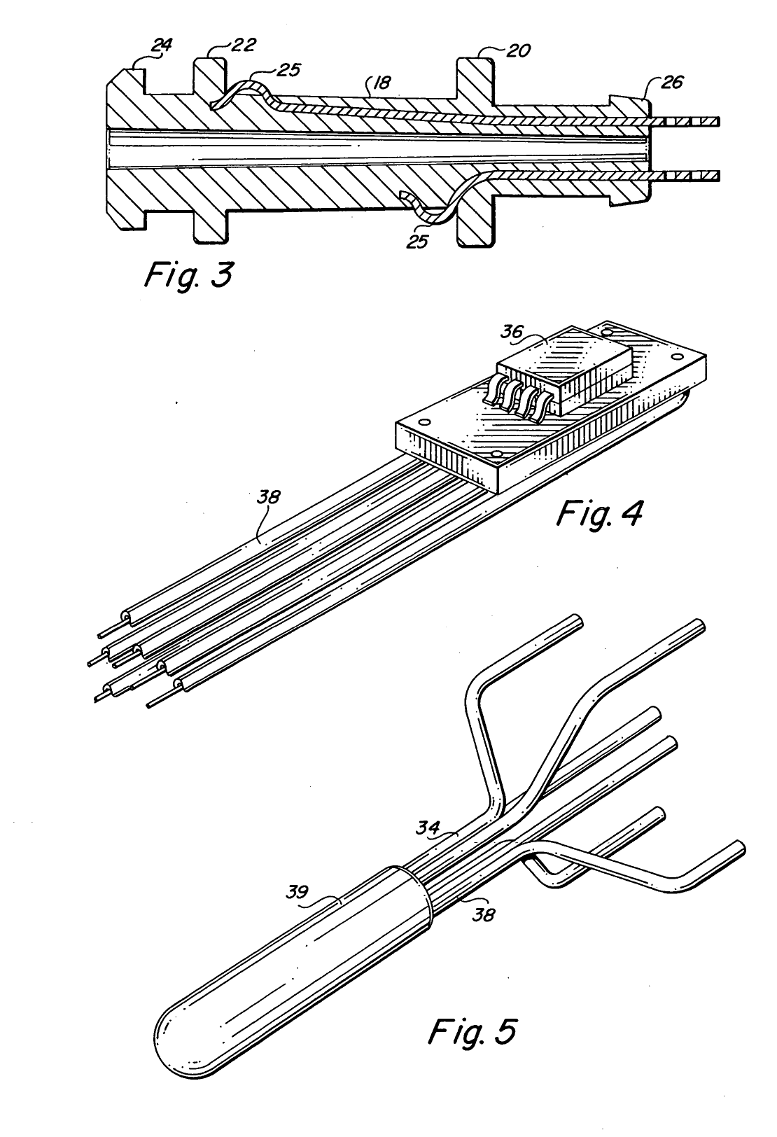

Referring to FIGS. 1 and 2, a small serial memory chip 36 is disposed near the proximal end 12 of the tube 10. The memory 36 is further attached to electrical wire 38 that leads to a RJ-45 connector. Referring to FIG. 3, the memory 36 and the electrical wires 38 are soldered to a printed circuit board and encapsulated in a nonconductive epoxy coating 39 (FIG. 4). The printed circuit board upon which the memory 36 is mounted is preferably no more than generally 0.125 inch by 0.562 inch and more preferably smaller than such dimensions.

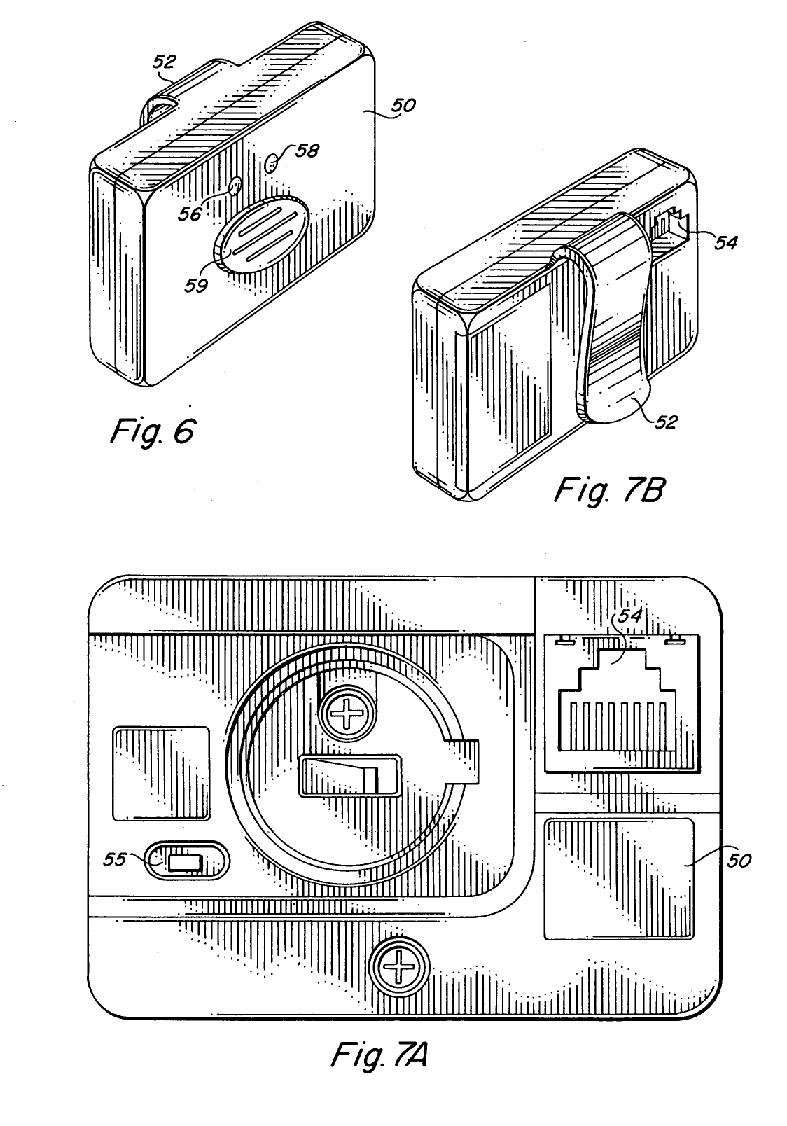

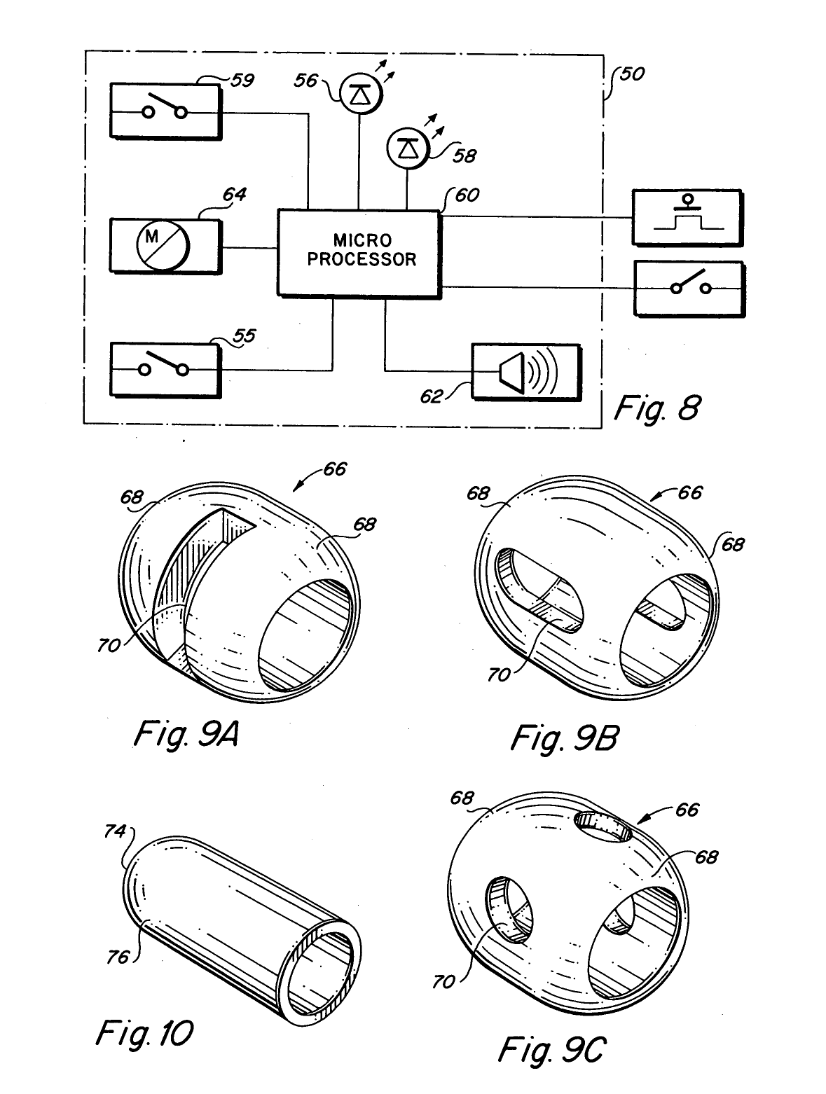

Referring to FIGS. 6, 7A, and 7B, the RJ-45 connector 40 is attached to a controller 50. The controller 50 comprises a small plastic box that may be attached to a user's belt or pant waistline by means of a clip 52 and further comprises a modular port 54 for insertion of and electrical connection with the RJ-45 connector 40. The controller 50 further comprises one green light emitting diode (“LED”) 58 and one red LED 56 that notify the user of the status of the electrical contacts 25, memory 36 and the controller 50. A switch 59 is provided so that the user may turn the controller on or off. A switch 55 is also provided so that the user may switch between vibrate and beep mode. Referring to FIG. 8, the controller 50 further comprises a microprocessor 60 electrically connected to the memory 36 and the electrical contacts 25. The microprocessor 60 is preferably a CY8C26233-24PVI made by the Cypress Microsystems Corp. of Bothell, Wash. The serial EEPROM is preferably an AT93C46 made by the Atmel Corp. of San Jose, Calif. During operation, the microprocessor 60 occasionally lights the green LED 58 to let the user know that the controller 50 is operating properly. When a low battery condition is present, the microprocessor 60 occasionally lights the red LED 56 to let the user know that the battery (not shown) is low and should be replaced soon.

When the presence of fecal matter near the probe 18 is indicated by electric current traveling between the metal contacts, the microprocessor 60 causes a vibrator motor 64 to operate if the controller is in vibration mode, or a speaker 62 to sound an audible tone if the controller is in beep mode, to notify the user that he/she should remove the catheter at the next opportunity to expel accumulated feces. To acknowledge and end the alert, the user presses the switch 59.

Referring to FIGS. 2, 9A, 9B, and 9C, in order to control how fecal matter reaches to the contacts, a fecal guard 66 is placed over the probe 18. The fecal guard 66 is maintained in position over the probe 18 by sliding a fecal guard 66 onto the probe 18 over the rigid, annular rib 24 and placing it over the journals 20, 22. The fecal guards 66 are generally cylindrical in shape with tapered ends 68. In a central region thereof, the fecal guards 66 comprise cutout portions 70 which may vary in size. By varying the size of the cutout portions 70, the ability of feces of different consistency to effectively reach the moisture detector can be varied.

Furthermore, referring to FIG. 9, a filter tip 72 is resiliently installed over the barbed rib 24 and held in place by tension and friction. The filter tip also secures the fecal guard 66 to the probe 18. Filter tip 72 has a tip 74 in the general shape of a paraboloid of revolution and has a hollow, cylindrical body 76.

Upon initial communication, the microprocessor 60 functions by polling the memory 36 to determine the present numeric value stored therein. New catheters are provided to the user with the numeric value set to 0. The microprocessor 60 then increments the value of the memory 36 at set time intervals. In this manner, by reading and incrementing the memory 36, the microprocessor 60 can identify how long the catheter 10 has been in use. The microprocessor 60 can further monitor the memory 36 to determine when the catheter has reached a point beyond which it is no longer safe to use. At such point, the microprocessor 60 can cease to function until a new catheter with a memory 36 value that has not reached an expiration point is attached, can continue to function during a grace period of operation before the microprocessor 60 discontinues operation or can continue indefinitely during the expired period. Preferably, the microprocessor 60 provides a notification to the user or the caretaker when the catheter has expired or will soon expire by lighting one or a combination of multiple LEDs and/or providing audible and/or tactile alerts. Upon expiration, the catheter 10 would be discarded and a new catheter with a new memory 36 set to zero would be implemented.

In use, the catheter 10 is inserted into the rectum and a balloon 16 on the end of the catheter is inflated through the air supply tube 28 to hold the catheter 10 in place and to seal the rectum. The catheter 10 is inflated by attachment of the check valve 30 to a syringe which forces a measured quantity of air into the balloon 16 to inflate it. When fecal matter is detected, the controller 50 alerts the user by an audible or tactile alert. The user may then stop the alert by pressing the switch 59, going to a restroom, deflating the balloon 16 by reinstalling the syringe and removing air, and removing the catheter 10 to allow the fecal matter to be expelled.

Alternatively, rather than providing a memory 36 within the catheter 10 that is incremented, the present invention could implement a memory having a numeric serial number stored thereon. The microprocessor 60 would include a memory that associated a count with the serial number, the count being incremented at predetermined intervals. When the count reached a predetermined threshold, the catheter associated with the serial number would be marked as expired within the memory of the controller and would either cease to function until a new catheter with an unexpired serial number was attached, continue to function during a grace period of operation before the controller discontinues operation with the expired catheter or continue indefinitely during the expired period. Furthermore, the controller 50 would be able to track multiple serial numbers of catheters that have been used with the controller 50, as well as store the periods of time such catheters were used with the controller 50.

In another alternative, the microcontroller could increment the count within the memory 36 not based on time, but instead based upon the occurrence of an event. For example, the memory 36 could be incremented every time an indication of moisture was detected by the moisture sensor 32. In the event of a catheter that could be used only once, the memory would only need to store a value indicating whether the catheter had detected a single moisture event.

Furthermore, it is envisioned that a radio frequency transmitter could be provided within the controller 50 to occasionally broadcast a signal to a receiving controller that allows a third party to remotely be notified of a signal from the moisture sensor or be notified of an expired or soon-to-expire condition of the catheter or any other information tracked by the controller.

Furthermore, it is envisioned that the controller could monitor an air pressure within the air supply tube 28 to determine the inflation status of the balloon 16. If the controller 50 detected that the balloon 16 was becoming deflated based upon a low air pressure reading, the controller 50 would alert the user or a caretaker so that appropriate action could be taken. It is also envisioned that an electrically operated air pump could also be provided within the controller to reinflate a balloon that, based on the air pressure sensor, was becoming deflated.

Furthermore, while EEPROM memory is shown and described, any memory capable of holding a value and being incremented could be used instead. Obviously, while the connector is described as a RJ-45 connector, any connector having an appropriate number of conductors would work equally well. Additionally, while it is described that the memory is incremented until an expiration point is reached and new catheters are provided with memory having a value set equal to zero, it is readily apparent to one of ordinary skill in the art that memory could be provided with a value equal to a predetermined value and wherein that value is decremented to zero by the microprocessor.

While the specific embodiments have been described, numerous modifications come to mind without significantly departing from the spirit of the invention, and the scope of protection should only limited by the scope of the accompanying claims.

1. A device for blocking an opening in the human body comprising:
a disposable portion comprising:
a probe capable of insertion into the opening that is adapted to detect a quantity of effluent;
an inflatable balloon for blocking the opening;
a memory associated with the probe for storing a value representing an amount of time that the probe has been in use;
a processor electrically connected to the catheter for reading and updating the value stored within the memory and communicating with the effluent detector to determine whether an effluent is present near the probe.

2. The device of claim 1 wherein the effluent detector within the probe comprises two contacts having a potential difference such that when moisture is present electrical current passes between the contacts through the moisture.

3. The device of claim 1 wherein the processor further monitors an air pressure within the inflatable balloon and provides an alert when the air pressure drops below a predetermined value.

4. The device of claim 1 further comprising an air pump and wherein the processor further monitors an air pressure within the inflatable balloon and operates the pump when the air pressure drops below a predetermined value.

5. The device of claim 1 wherein the processor alerts a user of the device of the presence of an effluent by a tactile alert.

6. The device of claim 1 wherein the processor alerts a user of the device of the presence of an effluent by an audible alert.

7. The device of claim 1 wherein the processor alerts a user of the device of the presence of an effluent by a visual alert.

8. The device of claim 1 wherein the memory is coated in a generally nonconductive protective coating.

9. The device of claim 1 wherein the probe further comprises a guard which controls the ability of a fluid to reach the contacts.

10. The device of claim 1 wherein the microprocessor will no longer communicate with the probe to determine whether an effluent is present near the probe after a predetermined value stored within the memory is reached.

11. The device of claim 1 wherein the microprocessor will provide an alert after a predetermined value stored within the memory is reached.

12. The device of claim 1 further comprising a RF transmitter for transmitting to an alert to a caretaker.

13. A medical device for detecting moisture in the human body comprising:
a disposable portion comprising:
a probe capable of insertion into the opening comprising a moisture sensor;
a memory associated with the probe for storing a value representing an amount of time that the probe has been in use;
a processor electrically connected to the catheter for reading and updating the value stored within the memory and communicating with the moisture sensor to determine whether moisture is present near the probe.

14. The device of claim 13 wherein the moisture sensor within the probe comprises two contacts having a potential difference such that when moisture is present electrical current passes between the contacts through the moisture.

15. The device of claim 13 wherein the processor alerts a user of the device of a moisture condition by at least one of a tactile alert, an audible alert and a visual alert.

16. The device of claim 13 wherein the memory is coated in a generally nonconductive protective coating.

17. The device of claim 13 wherein the probe further comprises a guard which controls the ability of a fluid to reach the contacts.

18. The device of claim 13 wherein the microprocessor will no longer communicate with the moisture sensor to determine whether moisture is present near the probe after a predetermined value stored within the memory is reached.

19. The device of claim 13 wherein the microprocessor will provide an alert after a predetermined value stored within the memory is reached.

20. The device of claim 13 further comprising a RF transmitter for transmitting to an alert to a caretaker.

21. A reusable medical device comprising:
a disposable portion comprising:
a probe capable of insertion into an opening of the human body for detecting a condition within the body;
a memory associated with the probe for storing a value representing an amount of time that the probe has been in use;
a processor electrically connected to the disposable portion for reading and updating the value stored within the memory and alerting the human or a caretaker of the presence of the condition.

22. The device of claim 21 wherein the memory is updated to represent the number of times the device has detected the presence of the condition.

23. The device of claim 22 wherein the microprocessor will no longer alert the user or caretaker when a predetermined number of times the device has detected the presence of the condition has been reached.

24. The device of claim 21 wherein the microprocessor will no longer alert the user or caretaker when a predetermined amount of time has been reached.

25. The device of claim 21 wherein the microprocessor will provide an alert after a predetermined value stored within the memory is reached.

26. The device of claim 22 wherein the microprocessor will provide an alert after a predetermined value stored within the memory is reached.
